(19) United States
(12) Patent Application Publication
(10) Pub. No.: US 2026/0363804 A1
(43) Pub. Date: Nov. 19, 2026

(54) SURGICAL DEVICE, SURGICAL METHOD, AND SURGICAL APARATUS

(71) Applicants: Beijing Great Robotics Technology Limited, Beijing (CN); Suzhou Great Robotics Medical Technology Co., Ltd., Suzhou, Jiangsu (CN)

(72) Inventor: Shang GAO, Beijing (CN)

(73) Assignees: Beijing Great Robotics Technology Limited, Beijing (CN); Suzhou Great Robotics Medical Technology Co., Ltd., Suzhou, Jiangsu (CN)

(21) Appl. No.: 19/133,710

(22) PCT Filed: Nov. 29, 2022
(86) PCT No.: PCT/CN2022/134887

§ 371 Date: Jul. 28, 2025

(51) Int. Cl. A61B 34/37; A61B 1/00; A61B 1/018; A61B 34/00; A61B 34/30; A61B 90/00; A61M 25/01

(52) U.S. Cl. CPC A61B 34/37; A61B 1/00149; A61B 1/018; A61B 34/25; A61B 2034/301; A61B 2090/0804; A61M 25/0113

(57) ABSTRACT

The present application provides a surgical device. The surgical device includes a robotic manipulator and a control apparatus. The robotic manipulator is provided with a surgical instrument assembly including multiple surgical instruments arranged on a switching apparatus. The control apparatus determines a specified surgical instrument required for a surgical operation, generates an instrument switching instruction according to the specified surgical instrument, and sends the instrument switching instruction to the robotic manipulator, and the robotic manipulator controls, according to the instrument switching instruction, the switching apparatus to move to switch the specified surgical instrument located at a to-be-selected position to an operation position and then controls the specified surgical instrument to perform the surgical operation according to a control instruction sent by the control apparatus.

### CROSS REFERENCE TO RELATED APPLICATION

This application is the national phase of International Application No. PCT/CN2022/134887 filed on Nov. 29, 2022, the disclosure of which is hereby incorporated by reference in its entirety.

### TECHNICAL FIELD

The present specification relates to the field of medical technologies, and in particular, to a surgical device, a surgical method and a surgical apparatus.

### BACKGROUND

With the development of minimally invasive surgical techniques, requirements for minimally invasive and precise surgical operations are increasingly high. Among them, spinal endoscope minimally invasive surgery, as an early developed minimally invasive technique, has been widely applied clinically. Compared with traditional open surgery, the spinal endoscope minimally invasive surgery has advantages of small trauma, less bleeding, quick recovery, exact curative effects and the like. Similar minimally invasive techniques, for example, neuroendoscope minimally invasive surgery, have also been widely used clinically, playing an important role in surgery such as pituitary adenoma and skull base glioma in the field of neurology, and at the same time, having the above similar minimally invasive effects.

In the prior art, in order to reduce the difficulty of manual surgery by an operator, a surgical device can be used to assist the operator in performing surgical operations, thereby reducing the uncertainty of manual operations during the surgery, and relieving the burden of the operator in the surgery while improving the safety of the surgery.

Although the current surgical device can alleviate the burden on the operator to a certain extent, for surgical scenarios that requires a large number of surgical instruments, the operator needs to manually replace the surgical instruments on the surgical device, increasing the complexity of the surgical operations.

### SUMMARY

The present specification provides a surgical device, a surgical method and a surgical apparatus to partially solve the above problems existing in the prior art.

This specification adopts the following technical solutions.

The present specification provides a surgical device, including a robotic manipulator 1 and a control apparatus 2, where the robotic manipulator 1 is provided with a surgical instrument assembly 11 including multiple surgical instruments 111 arranged on a switching apparatus 112;
the control apparatus 2 is configured to determine a specified surgical instrument 111 required for a surgical operation, generate an instrument switching instruction according to the specified surgical instrument 111, and send the instrument switching instruction to the robotic manipulator 1; generate and send a control instruction according to a position of the specified surgical instrument 111;
the robotic manipulator 1 is configured to, in response to the instrument switching instruction sent by the control apparatus 2, control the surgical instrument assembly 11 to move to a specified position, and control the switching apparatus 112 to move to switch the specified surgical instrument 111 located at a to-be-selected position in the surgical instrument assembly 11 to an operation position; in response to the control instruction sent by the control apparatus 2, control the surgical instrument assembly 11 to move to a surgical position, and perform a surgical operation corresponding to the control instruction through the specified surgical instrument 111.

In an implementation, a switching manner in which the switching apparatus 112 switches the specified surgical instrument 111 from the to-be-selected position to the operation position includes at least one of rotation or translation.

In an implementation, the switching apparatus 112 further includes an operation component 1121;
in response to an operation action input by an operator through the operation component 1121, the switching apparatus 112 is controlled to move.

In an implementation, the robotic manipulator 1 is further provided with an auxiliary instrument assembly 12 including at least a working cannula 121 and an endoscope 122;
a mounting position of the working cannula 121 on the robotic manipulator 1 is located on a first axis, a mounting position of the endoscope 122 on the robotic manipulator 1 is located on a second axis, and a mounting position of the surgical instrument 111 on the robotic manipulator 1 is located on a third axis;
the first axis, the second axis, and the third axis are coincident or parallel to each other.

In an implementation, when the first axis, the second axis, and the third axis are parallel to each other, a distance between any two axes of the first axis, the second axis, and the third axis is not greater than a preset threshold.

In an implementation, the switching apparatus 112 is uniformly provided with multiple instrument mounting grooves, each of which is mounted with at least one surgical instrument 111; one of the instrument mounting grooves arranged on the switching apparatus 112 is located at the operation position, and the rest of the instrument mounting grooves are located at to-be-selected positions.

In an implementation, the control apparatus 2 is configured to, when the specified surgical instrument 111 and a current surgical instrument 111 are mounted on a same instrument mounting groove, generate the instrument switching instruction according to mounting positions of the specified surgical instrument 111 and the current surgical instrument 111 respectively on the instrument mounting groove, and send the instrument switching instruction to the robotic manipulator 1.

In an implementation, the control apparatus 2 is configured to determine a control parameter of the switching apparatus 112 according to the to-be-selected position of the specified surgical instrument 111 and the operation position, and generate the instrument switching instruction according to the control parameter, where the control parameter includes at least one of a moving direction, a moving distance, or a rotation angle.

In an implementation, the surgical device further includes a positioning apparatus 3, where a first positioning mark 13 is arranged on the surgical instrument 111, and a second positioning mark 5 is arranged within a preset range of a diseased part of a patient;
the positioning apparatus 3 is configured to collect positions of the first positioning mark 13 and the second positioning mark 5 and send the positions to the control apparatus;
the control apparatus 2 is configured to determine, according to the position of the second positioning mark 5, the specified position that allows each surgical instrument 111 in the surgical instrument assembly 11 to be switched, and the surgical position that allows each surgical instrument 111 to perform the surgical operation.

In an implementation, the surgical device further includes a scanning apparatus 4, where a third positioning mark 41 is arranged on the scanning apparatus 4;
the scanning apparatus 4 is configured to scan the patient and obtain a scanning result of the diseased part of the patient, and send the scanning result to the control apparatus 2;
the positioning apparatus 3 is configured to collect and send a position of the third positioning mark 41;
the control apparatus 2 is configured to determine a relative position between the scanning apparatus 4 and the patient according to the position of the second positioning mark 5 and the position of the third positioning mark 41; and register the scanning result according to the relative position between the scanning apparatus 4 and the patient.

In an implementation, the robotic manipulator 1 further includes a fixed base 14; the surgical instrument assembly 11 further includes a sliding block 113 and a clamping apparatus 114; where the clamping apparatus 114 clamps the switching apparatus 112, the clamping apparatus 114 is slidably connected to the sliding block 113, and the sliding block 113 is slidably connected to the fixed base 14;
the sliding block 113 is configured to, when sliding on the fixed base 14, drive the clamping apparatus 114 to slide along an extending direction of the fixed base 14 so as to drive the surgical instrument 111 to slide along the extending direction of the fixed base 14;
the clamping apparatus 114 is configured to, when sliding on the sliding block 113, drive the surgical instrument 111 to slide along the extending direction of the fixed base 14.

In an implementation, the control apparatus 2 is configured to determine, according to positions of the surgical instrument assembly 11 and a diseased part of a patient, a surgical path for the surgical instrument 111 to perform the surgical operation on the diseased part; generate a moving instruction according to the surgical path, and send the moving instruction to the robotic manipulator 1;
the robotic manipulator 1 is configured to move according to a received moving instruction to enable the extending direction of the fixed base 14 to be parallel to the surgical path, so that the surgical instrument 111 reaches the diseased part along the surgical path.

In an implementation, the control apparatus 2 further includes a human-computer interaction unit 21;
the human-computer interaction unit 21 is configured to, in response to an operation of an operator, determine the specified surgical instrument 111.

In an implementation, the human-computer interaction unit 21 includes a touch screen 211 and/or a control rod 212.

In an implementation, the surgical device further includes a base 6 and a suspension platform 61, where a head end of the suspension platform 61 is mounted on the base 6; the robotic manipulator 1 is hoisted at a tail end of the suspension platform 61.

In an implementation, the surgical device further includes a base 6, where the robotic manipulator 1 is mounted on the base 6.

The present specification provides a surgical method, where the method is applied to a control apparatus and includes:
determining a specified surgical instrument required for a surgical operation;
generating an instrument switching instruction according to the specified surgical instrument, and sending the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position; and
generating and sending a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

The present specification provides a surgical apparatus, where the apparatus is applied to a control apparatus and includes:
a determining module, configured to determine a specified surgical instrument required for a surgical operation;
an instrument switching instruction generating module, configured to generate an instrument switching instruction according to the specified surgical instrument, and send the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position; and
a control instruction generating module, configured to generate and send a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

The present specification provides a computer-readable storage medium, where the storage medium stores a computer program, and when the computer program is executed by a processor, the surgical method as described above is implemented.

At least one of the above technical solutions adopted in the present specification can achieve the following beneficial effects.

The present specification provides a surgical device. The surgical device includes a robotic manipulator and a control apparatus. The robotic manipulator is provided with a surgical instrument assembly including multiple surgical instruments arranged on a switching apparatus. The control apparatus determines a specified surgical instrument required for a surgical operation, generates an instrument switching instruction according to the specified surgical instrument, and sends the instrument switching instruction to the robotic manipulator, and the robotic manipulator controls, according to the instrument switching instruction, the switching apparatus to move to switch the specified surgical instrument located at a to-be-selected position to an operation position and then controls the specified surgical instrument to perform the surgical operation according to a control instruction sent by the control apparatus. It can be seen that rapid switching between multiple surgical instruments is realized by the surgical device, which can eliminate the operations for operators to manually replace the surgical instruments during surgery in surgical scenarios requiring a large number of surgical instruments, further improving the precision of surgical operations and relieving the operation burden on operators.

### BRIEF DESCRIPTION OF THE DRAWINGS

The drawings described herein are used to provide a further understanding of the present specification, and constitute a part of this specification, and the schematic embodiments of the specification and the description thereof are used to explain the present specification, but do not constitute an improper limitation on the specification.

FIG. 1A is a schematic diagram of a surgical device in the present specification.

FIG. 1B is a schematic diagram of a surgical device in the present specification.

FIG. 2A is a schematic diagram of a surgical device in the present specification.

FIG. 2B is a schematic diagram of a surgical device in the present specification.

FIG. 2C is a schematic diagram of a surgical device in the present specification.

FIG. 2D is a schematic diagram of a surgical device in the present specification.

FIG. 3A is a schematic diagram of a surgical device in the present specification.

FIG. 3B is a schematic diagram of a surgical device in the present specification.

FIG. 3C is a schematic diagram of a surgical device in the present specification.

FIG. 4 is a schematic diagram of a surgical device in the present specification.

FIG. 5 is a schematic diagram of a surgical device in the present specification.

FIG. 6A is a schematic diagram of a surgical device in the present specification.

FIG. 6B is a schematic diagram of a surgical device in the present specification.

FIG. 7A is a schematic diagram of a surgical device in the present specification.

FIG. 7B is a schematic diagram of a surgical device in the present specification.

FIG. 7C is a schematic diagram of a surgical device in the present specification.

FIG. 7D is a schematic diagram of a surgical device in the present specification.

FIG. 8 is a schematic diagram of a control apparatus in the present specification.

FIG. 9 is a schematic diagram of a surgical device in the present specification.

FIG. 10 is a schematic flowchart of a surgical method in the present specification.

FIG. 11 is a schematic diagram of a surgical apparatus provided in the present specification.

### DETAILED DESCRIPTION OF THE EMBODIMENTS

In order to make the objectives, technical solutions and advantages of the present specification clearer, the technical solutions of the present specification will be clearly and completely described below with reference to specific embodiments of the present specification and corresponding drawings. Obviously, the described embodiments are only some but not all of the embodiments of the specification. All other embodiments obtained by those skilled in the art based on the embodiments of the present specification without creative efforts shall fall within the protection scope of the present specification.

In addition, it should be noted that all actions of acquiring signals, information or data in the present disclosure are performed under the premise of complying with corresponding data protection regulation policy of local country, and obtaining authorization given by an owner of a corresponding apparatus.

Currently, regardless of orthopedics, neurology, intervention and the like, treatment can be carried out through minimally invasive surgery, so that requirements for minimally invasive and precise surgical operations are increasingly high, and it is necessary to try to achieve minimal trauma and accurately reach a diseased part for effective treatment. Among them, spinal endoscope minimally invasive technique, as an early developed minimally invasive technique, causes less injury to paravertebral muscles, and can keep a complete spine structure to the greatest extent, maintaining the postoperative stabilization of spine, and having the advantages of small trauma, less bleeding, quick recovery, few complications, exact curative effects and the like. Minimally invasive techniques, similar to the spinal endoscope minimally invasive technique, further include neuroendoscope minimally invasive technique, which has also been widely used clinically, playing an important role in surgery such as pituitary adenoma and skull base glioma in the field of neurology, and at the same time, having the above similar minimally invasive effects.

For example, currently using a minimally invasive surgical device to assist an operator (such as a doctor) in performing a surgical operation has been widely used in the field of spinal endoscope minimally invasive surgery, and there are two main surgical manners commonly used in the spinal endoscope minimally invasive surgery: a Percutaneous Endoscopic Lumbar Discectomy (PELD) and a Unilateral Biportal Endoscopy (UBE), for both of which auxiliary operations can be performed by the minimally invasive surgical device. However, the fundamental purpose of using a minimally invasive surgical table to assist the operator in performing the surgical operation is to relieve the operation burden on the operator during surgery, and now, in a complex surgical scenario requiring a large number of surgical instruments, the operator still needs to manually replace the surgical instruments on the surgical device during the surgery, increasing the complexity of the surgical operation.

Based on this, the present specification provides a surgical device, where a control apparatus sends an instrument switching instruction to a robotic manipulator, so that the robotic manipulator controls switching between surgical instruments arranged on a switching apparatus according to the instrument switching instruction, realizing the rapid switching between multiple surgical instruments, eliminating the operation for operators to manually replace the surgical instruments during surgery, further improving the precision of surgical operations and relieving the operation burden on operators.

The technical solutions provided in the embodiments of the present specification will be described in detail below with reference to the accompanying drawings.

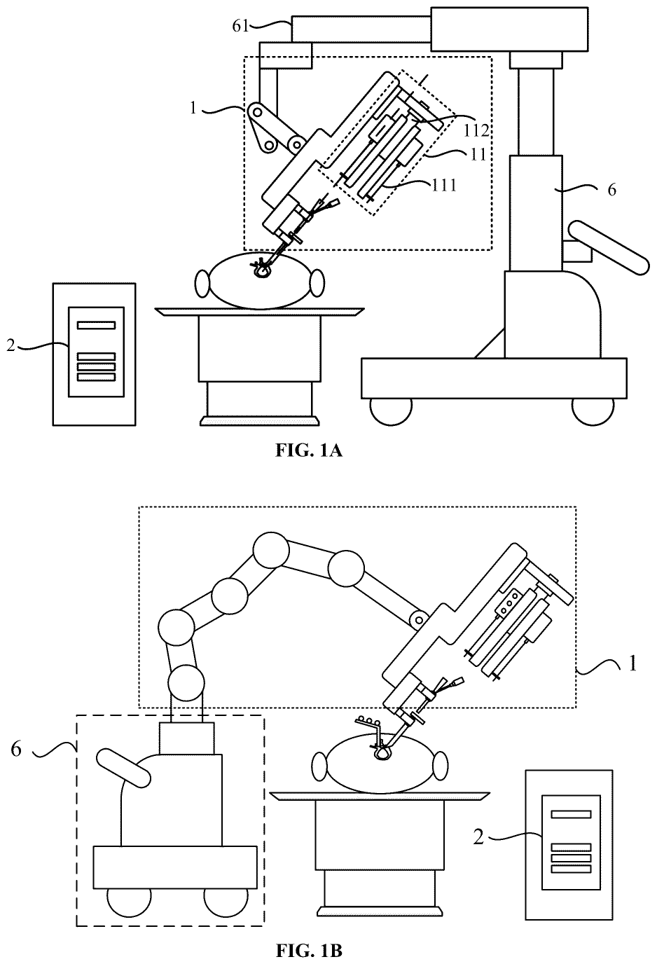

FIG. 1A is a schematic diagram of a surgical device provided in the present specification.

The present specification provides a surgical device, which can assist an operator in performing a surgical operation. In the embodiments of the present specification, for ease of understanding, specific technical solutions will be described by taking spinal endoscope surgery as an example.

As shown in FIG. 1A, the surgical device includes at least a robotic manipulator 1 and a control apparatus 2, where the robotic manipulator 1 is provided with a surgical instrument assembly 11 including multiple surgical instruments 111 arranged on a switching apparatus 112. The control apparatus 2 is configured to send a control instruction to the robotic manipulator 1, and the robotic manipulator 1 is configured to control the surgical instruments 111 to perform multiple different types of actions according to the control instruction. According to different application scenarios, the control instruction sent by the control apparatus 2 may be preset and stored in the control apparatus 2, or be generated by the control apparatus 2 in response to an operation action input by an operator.

The robotic manipulator 1 may be mounted on a base 6, and a mounting manner thereof may be a suspension manner (as shown in FIG. 1A), or be a floor manner (as shown in FIG. 1B), or be any existing mounting manner, which is not limited in this specification. In order to ensure the flexibility of the robotic manipulator 1, a robotic manipulator 1 with six or more movable joints may be employed. The control apparatus 2 may be a computer host equipped with specified software (such as three-dimensional software, planning software), and is connected to at least the robotic manipulator 1 in a wired or wireless manner. The control apparatus 2 may be arranged in a surgical environment where the robotic manipulator 1 is located, or in other device environments, which is not limited in this specification.

In an implementation, the base 6 where the robotic manipulator 1 is located may be temporarily fixed on the floor, or be equipped with movable pulleys, so as to move according to actual needs, examples as shown in FIG. 1A and FIG. 1B are merely placement examples of the base in the embodiments of the present specification, and an actual position thereof may be set according to an application scenario.

The robotic manipulator 1 is provided with at least the surgical instrument assembly 11. In practical applications, facing different disease conditions (such as herniation of nucleus pulposus, spinal stenosis, nerve root compression, dural sac, and bone tissue hyperplasia), surgical instruments 111 to be adopted are different, and different disease conditions may need to be treated in a surgical process of a patient. Performing multiple manual replacements of a single surgical instrument 111 arranged on the robotic manipulator 1 during surgery not only increases a surgical time, but also increases an operation difficulty of an operator. Based on this, in one or more embodiments of the present specification, the surgical instrument assembly 11 arranged on the robotic manipulator 1 may include multiple surgical instruments 111. Therefore, the operator may arrange multiple surgical instruments 111 in the surgical instrument assembly 11 according to actual conditions of a diseased part of a patient, and switch the surgical instruments 111 by the robotic manipulator 1 in response to an instrument switching instruction, in which way, the requirement of using different surgical instruments 111 to perform different surgical operations (such as remove herniated tissues, remove bone substance, and repair damaged annulus fibrosus with a radio-frequency electrode) in a surgical process are met.

The surgical instrument assembly 11 includes a switching apparatus 112, and several surgical instruments 111 may be mounted on the switching apparatus 112. The switching apparatus 112 may switch between the surgical instruments 111 by moving itself, so as to switch a specified surgical instrument 111 required for a surgical operation from a to-be-selected position to a surgical position. A movement manner of the switching apparatus 112 may be rotation, translation, or a combination thereof, and of course, may further be any existing movement manner, which is not limited in this specification. The surgical instrument assembly 11 may include one or more surgical instruments 111 for a spinal minimally invasive surgical operation, such as nucleus pulposus forceps, electrode electrocoagulation, a microsurgical scalpel, microforceps, and a microwave ablation needle. The specific types of the surgical instruments 111 can be configured according to specific application scenarios, which is not limited in this specification. In the embodiments of the present specification, the operation position refers to a position of a surgical instrument 111 on the switching apparatus 112 where a surgical operation can be performed, and relatively, the to-be-selected position refers to other positions on the switching apparatus 112 except the operation position.

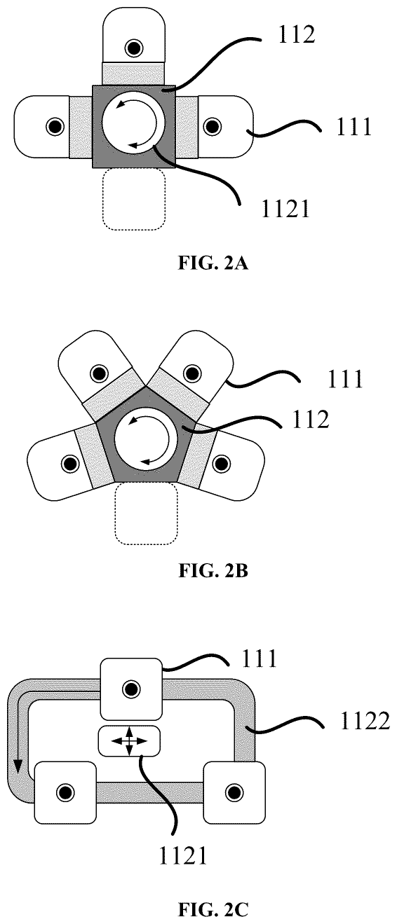

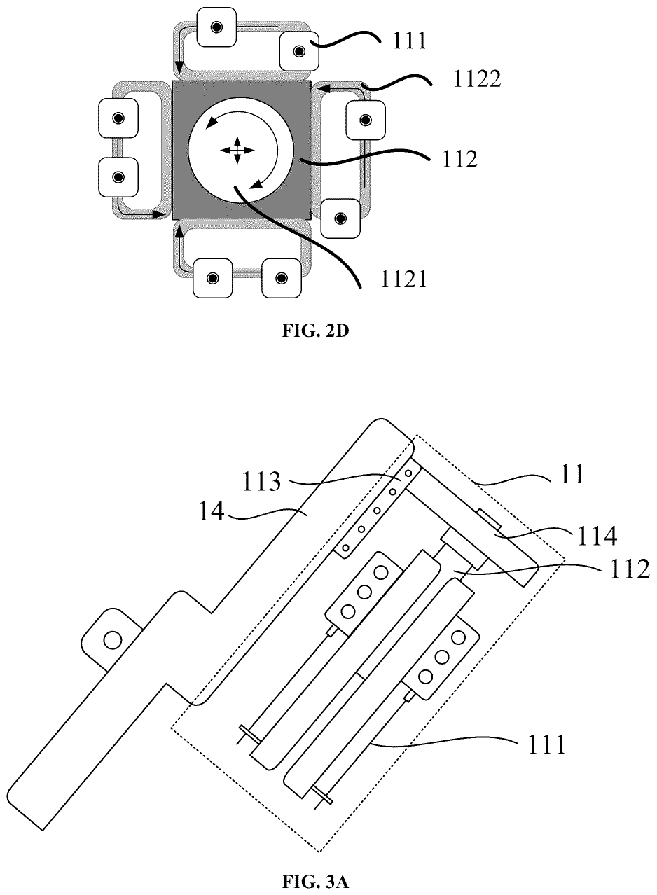

Specifically, the switching apparatus 112 in the surgical instrument assembly 11 may be a member having at least two surgical instrument mounting surfaces, each mounting surface may be provided with one instrument mounting groove, and each instrument mounting groove may be mounted with at least one surgical instrument 111. In addition, timing of mounting the surgical instrument 111 on the switching apparatus 112 may be preoperative or intraoperative, which is not limited in this specification. For example, as can be known from the cross-sectional view of the surgical instrument assembly 11 shown in FIG. 2A, the dotted portion is the instrument mounting groove where the surgical instrument 111 is not mounted temporarily, and an operator can disassemble and assemble the surgical instrument mounted on the switching apparatus 112 according to actual conditions.

In an implementation, if the switching manner in which the switching apparatus 112 switches the specified surgical instrument 111 from the to-be-selected position to the operation position is rotation, the switching apparatus 112 includes a columnar member provided with at least two surgical instrument mounting surfaces. Each surgical instrument 111 is mounted along an axis of the switching apparatus 112, and the axis of each surgical instrument 111 is parallel to the axis of the switching apparatus 112. The robotic manipulator 1 is configured to, in response to the instrument switching instruction, control the switching apparatus 112 to rotate about its own axis to switch the specified surgical instrument 111 from the to-be-selected position to the operation position. For example, when the switching manner of the switching apparatus 112 is rotation, the surgical instrument assembly 11 may be a switchable spiral structure encircling around the switching apparatus 112. In addition, the surgical instruments 111 may be an arrangement of two or more surfaces on the switching apparatus 112, such as an arrangement of four surfaces (as shown in FIG. 2A) and an arrangement of five surfaces (as shown in FIG. 2B), which is not limited in this specification.

In an implementation, if the switching manner in which the switching apparatus 112 switches the specified surgical instrument 111 from the to-be-selected position to the operation position is translation, the switching apparatus 112 includes a guide rail 1122 provided with at least two surgical instrument mounting surfaces. The surgical instruments 111 are mounted on the guide rail 1122 through the surgical instrument mounting surfaces, and move on the guide rail 1122. The robotic manipulator 1, in response to the instrument switching instruction, controls the guide rail 1122 to move to drive the specified surgical instrument 111 to be switched from the to-be-selected position to the operation position. It can be seen that, when the switching manner of the switching apparatus 112 is translation, the switching apparatus 112 may include the guide rail 1122. The guide rail 1122 may be an annular guide rail, a linear guide rail, or any geometric-shaped guide rail, which is not specifically limited in this specification. For example, the guide rail 1122 as shown in FIG. 2C is an annular guide rail, the surgical instruments 111 can be mounted on the guide rail 1122, and the surgical instruments 111 can move on the guide rail 1122 to realize switching between the surgical instruments 111.

In an implementation, if the switching manner in which the switching apparatus 112 switches the specified surgical instrument 111 from the to-be-selected position to the operation position is rotation and translation, the switching apparatus 112 includes a columnar member provided with at least two surgical instrument mounting surfaces, where each surgical instrument mounting surface is provided with a guide rail 1122, and the guide rail 1122 is mounted with at least one surgical instrument 111 which moves on the guide rail 1122. The robotic manipulator 1, in response to the instrument switching instruction, controls the switching apparatus 112 to rotate around its own axis toward a first direction corresponding to the instrument switching instruction, so as to switch the guide rail mounted with the specified surgical instrument 111 from the to-be-selected position to the surgical position; and controls the guide rail 1122 to move toward a second direction corresponding to the instrument switching instruction, so as to move the specified surgical instrument 111 to the surgical position. The guide rail 1122 may be an annular guide rail, a linear guide rail, or any geometric-shaped guide rail, which is not specifically limited in this specification. As shown in FIG. 2D, the guide rail 1122 is an annular guide rail. By means of rotation and translation, more surgical instruments can be mounted on the switching apparatus 112, which is more suitable for the surgical scenario requiring a large number of surgical instruments, improving the efficiency of surgery performed based on the surgical device, and reducing the complexity of surgical operations.

The three switching manners of the switching apparatus are only embodiments provided in this specification, and do not represent that the switching apparatus 112 is only of the above structures and can only realize switching in the above manners. In the embodiments of the present specification, the manner in which the switching apparatus 112 switches the surgical instrument may be rotation, translation, or a combination thereof, and of course, may further be any existing movement manner, which is not limited in this specification. The guide rail 1122 may be an annular guide rail, a linear guide rail, or any geometric-shaped guide rail, which is not specifically limited in this specification.

A mounting sequence and specific mounting positions of the surgical instruments 111 on the switching apparatus 112 are not limited in the embodiments of the present specification, and arrangement of the surgical instruments 111 is not necessarily uniform. The operator may arrange the multiple surgical instruments 111 included in the surgical instrument assembly 11 according to specific surgical needs, and specific arrangement manners of the surgical instruments 111 are not limited in the embodiments of the present specification.

In one or more embodiments of the present specification, the switching apparatus 112 is moved to realize switching between the surgical instruments 111 included in the surgical instrument assembly 11, which may be controlled in response to the instrument switching instruction sent by the control apparatus 2, or be manually controlled by the operator.

The instrument switching instruction sent by the control apparatus 2 may be sent by the operator by operating an operation control rod 2122 arranged on the control apparatus 2, or be sent according to a pre-planned surgical planning action on the diseased part by the surgical instrument 111, or be sent through an instrument switching option arranged in the control apparatus 2, which is not limited in this specification.

In an implementation, the operator manually controlling the switching apparatus 112 to move to switch the surgical instrument 111 may be implemented in the following manner. The switching apparatus 112 further includes an operation component 1121, and in response to an operation action input by the operator through the operation component 1121, the switching apparatus 112 is moved to move the specified surgical instrument 111 from the to-be-selected position to the surgical position. The operation component 1121 may be controlled to tilt toward multiple different directions to control the switching apparatus to translate toward multiple different directions, or be controlled to rotate to control the switching apparatus to rotate. The operation action input by the operator through the operation component 1121 may be at least one of rotation or translation. As shown in FIG. 2A, the operation component 1121 is a component in a rotation form, and as shown in FIG. 2C, the operation component is a component in a translation form. Of course, the operation component 1121 can realize control over both the rotation and translation of the switching apparatus 112, as shown in FIG. 2D.

It can be seen that rapid switching between multiple surgical instruments 111 is realized by the surgical device, which can eliminate the operations for operators to manually replace the surgical instruments 111 during surgery in surgical scenarios requiring a large number of surgical instruments 111, further improving the precision of surgical operations and relieving the operation burden on operators.

In addition, each surgical instrument 111 has at least three degrees of freedom, such as forward and backward movements, instrument opening and closing, and instrument rotation.

Based on the structure that multiple surgical instruments can be mounted on the switching apparatus 112, in an embodiment of the present specification, the control apparatus 2 may determine a control parameter of the switching apparatus 112 according to a difference between the to-be-selected position and the operation position, and generate the instrument switching instruction according to the control parameter. The control parameter may include a rotation direction, a rotation angle, a movement direction, and a movement distance. For example, taking the control parameter being the rotation direction and the rotation angle as an example, switching from the to-be-selected position to the operation position requires the switching apparatus 112 to rotate clockwise by 60°, then it is determined that the rotation direction is clockwise, and the rotation angle is 60°, based on which, the instrument switching instruction is generated. In an implementation, the rotation direction of the switching apparatus 112 may be a rotation direction with a smaller rotation angle when rotating from the to-be-selected position to the operation position. For example, switching from the to-be-selected position to the operation position requires the switching apparatus 112 to rotate clockwise by 60°, and relatively, switching from the to-be-selected position to the operation position may be rotating counterclockwise by 300°. The rotation direction with a smaller rotation angle is usually a preferred rotation direction, i.e., a clockwise rotation direction. Of course, determination of the rotation direction is merely a solution provided in the embodiments of the present specification, and a specific control parameter determining solution may be determined according to a specific application scenario, which is not limited in this specification.

In an embodiment of the present specification, the solution that the switching apparatus 112 may be provided with multiple surgical instruments 111 may be: the switching apparatus 112 is provided with multiple instrument mounting grooves, each of which is mounted with at least one surgical instrument 111. Specifically, in the instrument mounting grooves, there exists at least one instrument mounting groove located at the operation position, and the surgical instrument 111 that needs to perform a surgical operation is mounted in the instrument mounting groove located at the operation position, so that the surgical instrument 111 can perform the surgical operation at the operation position. In addition, the multiple instrument mounting grooves may be uniformly or non-uniformly arranged on the switching apparatus 112, which is not limited in this specification.

Based on the structure of the switching apparatus 112, in an implementation, when the switching apparatus 112 is provided with multiple instrument mounting grooves, if the instrument mounting groove where the determined specified surgical instrument 111 required for the surgical operation is located is located at the to-be-selected position, and in addition to the specified surgical instrument 111, other surgical instruments 111 are also mounted on the instrument mounting groove, the instrument switching instruction may be first generated according to a difference between the to-be-selected position and the operation position, so as to rotate and move the instrument mounting groove where the specified surgical instrument 111 is located to the operation position. Then, a control parameter of the switching apparatus 112 are determined according to mounting positions of the specified surgical instrument 111 and the other surgical instruments 111 respectively on the instrument mounting groove, and the instrument switching instruction is generated again to move the specified surgical instrument 111 to the operation position.

In an embodiment of the present specification, the instrument mounting groove may be provided with a first mounting identifier, and the surgical instrument 111 may be provided with a second mounting identifier, where a correspondence exists between the first mounting identifier and the second mounting identifier, and the correspondence may be determined before the surgical operation is performed. When the surgical instrument 111 is mounted on the switching apparatus 112 through the instrument mounting groove, the first mounting identifier of the instrument mounting groove may be matched with the second mounting identifier of the to-be-mounted surgical instrument 111, and only when the first mounting identifier is matched with the second mounting identifier, the to-be-mounted surgical instrument 111 can be mounted on the instrument mounting groove.

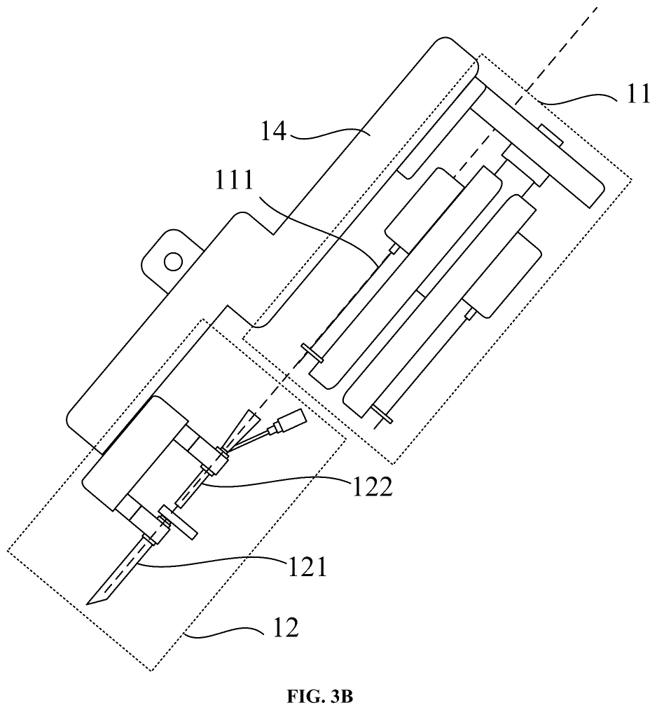

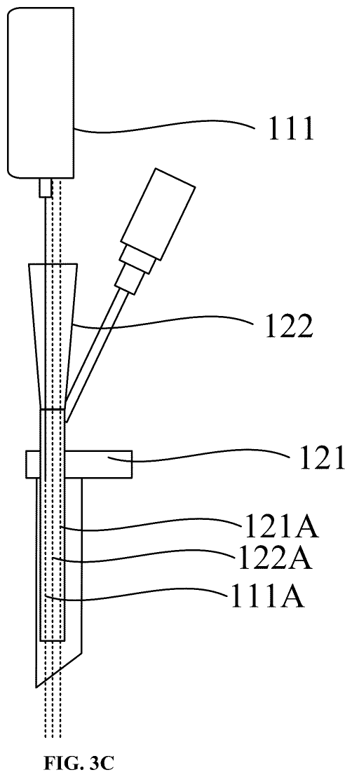

In an embodiment of the present specification, as shown in FIG. 3A, the robotic manipulator 1 includes a fixed base 14, and the surgical instrument assembly 11 is arranged on the fixed base 14. The surgical instrument assembly 11 further includes a sliding block 113 and a clamping apparatus 114, where the clamping apparatus 114 is configured to clamp the switching apparatus 112. It can be seen that, by moving the clamping apparatus, the switching apparatus 112 can be moved, and further the movement of the switching apparatus 112 can drive the surgical instrument 111 to move. In addition, the sliding block 113 is slidably connected to the fixed base 14, and the clamping apparatus 114 is slidably connected to the sliding block 113.

Based on the above structure, when the sliding block 113 slides on the fixed base 14, the clamping apparatus 114 is driven to slide along an extending direction of the fixed base 14 so as to drive the surgical instrument 111 to slide along the extending direction of the fixed base 14. When the clamping apparatus 114 slides on the sliding block 113, the surgical instrument 111 is driven to slide along the extending direction of the fixed base 14. It can be understood that the extending direction of the fixed base 14 refers to a direction parallel to an axis of the fixed base 14. It can be seen that, based on the above two-level sliding structure, when the extending direction of the fixed base 14 is aligned with a surgical path, the surgical instrument 111 can slide on the fixed base 14 through the sliding block 113, and/or slide on the sliding block 113 through the clamping apparatus 114, to reach a specified part of a patient to perform the surgical operation.

In an embodiment of the present specification, the robotic manipulator 1 is further provided with an auxiliary instrument assembly 12 including at least a working cannula 121 and an endoscope 122. The working cannula 121 is configured to dilate a puncturing wound, and establish a surgical channel from the outside to the diseased part of the patient to prevent the surgical instrument 111 from touching other parts where the surgical operation is not allowed; the endoscope 122 is configured to collect a real-time image of the diseased part to assist the surgical operation of the surgical instrument 111.

A mounting position of the working cannula 121 on the robotic manipulator 1 is located on a first axis, a mounting position of the endoscope 122 on the robotic manipulator 1 is located on a second axis, and a mounting position of the surgical instrument 111 that needs to perform the surgical operation on the robotic manipulator 1 is located on a third axis. A spatial position relationship among the first axis, the second axis, and the third axis may be a coincident relationship (as shown in FIG. 3B) or be a parallel relationship (as shown in FIG. 3C). Therefore, during the surgery, by aligning the axes of the working cannula 121, the endoscope 122, and the surgical instrument 111 that needs to perform the surgical operation with the planned surgical path, it is possible to ensure that the working cannula 121, the endoscope 122, and the surgical instrument 111 that needs to perform the surgical operation move along the surgical path without multiple alignments, reducing the difficulty of the surgical operation, and improving the efficiency and accuracy of the surgical operation.

In particular, as shown in FIG. 3C, when the spatial position relationship among the first axis, the second axis, and the third axis is a relationship of the three being parallel to each other, a distance between any two axes of the first axis, the second axis, and the third axis is not greater than a preset threshold. This is because the working channel established by the working cannula 121 has a certain width, and even if the second axis where the endoscope 122 is located does not coincide with the axis where the working cannula 121 is located, as long as the distance between the first axis and the second axis is not greater than the preset threshold, the endoscope 122, when being inserted into the working cannula 121, can enter the working cannula 121 without secondary alignment. The same applies to the endoscope 122 and the surgical instrument 111. As long as the distance between the second axis where the endoscope 122 is located and the third axis where the surgical instrument 111 is located is not greater than the preset threshold, the surgical instrument 111, when entering an instrument channel of the endoscope 122, can directly enter the instrument channel of the endoscope 122 without secondary alignment, improving the efficiency and accuracy of the surgical operation. Of course, by limiting the distance between the first axis and the third axis, whether the surgical instrument needs secondary alignment for entering the instrument channel of the endoscope 122 may be determined, that is, when the distance between the first axis and the third axis is not greater than the preset threshold, the surgical instrument 111 can directly enter the instrument channel of the endoscope 122 without secondary alignment. In addition, the first axis, the second axis and the third axis may or may not be on a same plane, which is not limited in this specification.

The preset threshold may be determined according to at least two of the size of the surgical instrument 111, the size of the instrument channel of the endoscope 122, the size of the endoscope 122 and the size of the working channel established by the working cannula 121, and of course, may also be preoperatively determined by the operator, which is not limited in this specification.

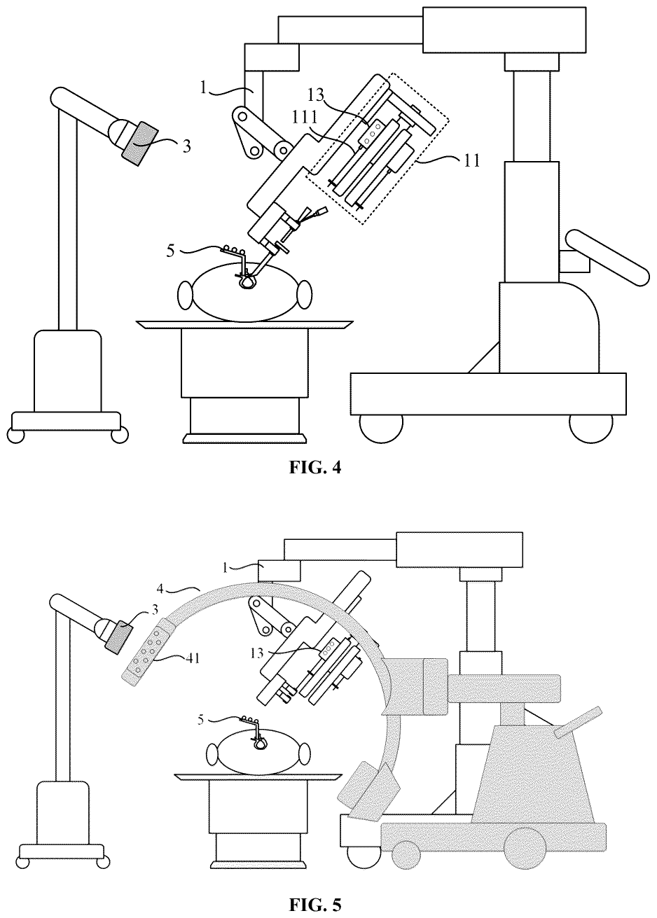

In addition, in an embodiment of the present specification, the surgical device further includes a positioning apparatus 3. As shown in FIG. 4, the positioning apparatus 3 may be mounted on a device located on a side of a surgical table, or be separately suspended above the patient (above the surgical table), or be arranged on any other device in a surgical environment, and be placed near the surgical table at a position suitable to collect positioning marks so as to acquire positions of positioning marks arranged near the diseased part of the patient, on the robotic manipulator 1, and on the scanning apparatus 4. The positioning marks may be optical positioning marks of light emitted or reflected by an infrared light emitting device, a Light Emitting Diode (LED), a passive reflecting sphere, or the like, and may also be other types of positioning marks. In the embodiments of the present specification, the positioning apparatus 3 captures the position of any device provided with a positioning mark by receiving the light emitted or reflected by the positioning mark.

In an embodiment of the present specification, the surgical device further includes a scanning apparatus 4, and the operator may scan the diseased part of the patient through the scanning apparatus 4 (such as intraoperative Computed Tomography (CT), or a 3-dimensional (3D) C-arm X-ray machine) in a surgical environment. The scanning apparatus 4 sends a scanning result to the control apparatus 2, which can simulate a three-dimensional image of the diseased part of the patient and upload the three-dimensional image to the control apparatus 2 for display so as to provide reference for the surgical operation of the operator. In addition, a third positioning mark 41 may be arranged on the scanning apparatus 4, so that the positioning apparatus 3 can determine the position of the scanning apparatus 4, and determine a relative position between the scanning apparatus 4 and the patient according to the position of the patient, as shown in FIG. 5.

A process of performing a surgical operation based on the surgical device will be described in detail below with reference to the accompanying drawings.

At a first step, before surgery is started, a patient is located on a surgical table in a prone pose, so that an operator and a surgical device perform a surgical operation on a diseased part of the patient.

At a second step, after the patient is confirmed to be in position, the diseased part of the patient is scanned by the scanning apparatus 4 to obtain a scanning result (an X-ray or CT image), and the scanning apparatus 4 may send the scanning result to the control apparatus 2 so as to provide reference and assistance for subsequent surgical operations.

In an embodiment of the present specification, a second positioning mark 5 is arranged within a preset range of the diseased part of the patient, and a third positioning mark 41 may be arranged on the scanning apparatus 4. A position of a first positioning mark 13 and the position of the third positioning mark 41 are collected and sent by the positioning apparatus 3. The control apparatus 2 is configured to determine a relative position between the scanning apparatus 4 and the patient according to the position of the second positioning mark 5 and the position of the third positioning mark 41, and register the scanning result according to the relative position between the scanning apparatus 4 and the patient. Further, a three-dimensional image of the diseased part of the patient in a coordinate system of a surgical environment is simulated according to the registered scanning result by three-dimensional software in the control apparatus 2, and is displayed on a display 22 of the control apparatus 2 so as to provide reference for the surgical operation of the operator.

At a third step, the control apparatus 2 further includes a planning module (not shown in the drawings), and according to the position of the diseased part of the patient determined by the positioning apparatus 3 and the registered scanning result of the diseased part, the planning module may plan the surgical path, the specified position that allows each surgical instrument 111 in the surgical instrument assembly 11 to be switched, and the surgical position that allows each surgical instrument 111 to perform the surgical operation. The surgical path includes at least a puncturing position and a puncturing direction when a working channel is established. Through the pre-planned surgical path, the specified position and the surgical position, the purpose of minimizing physical trauma to the patient while ensuring safe surgery is achieved.

In addition, when the surgery is performed on the patient, a region (such as nerve root, or spinal cord) near the diseased part of the patient tends to be very dangerous, and once an operation error occurs, it is extremely likely to cause other complications of the patient and even threaten the life safety of the patient. Therefore, according to the registered scanning result of the diseased part, the control apparatus 2 may further plan a safe surgery region that allows the surgical instrument 111 to perform the surgical operation as a specified region, and relatively, regions other than the specified region are delineated as dangerous regions, which are regions where the surgical instrument 111 is not allowed to perform any operation.

In an implementation, it is also possible to plan, based on the planned specified region, dangerous regions according to the position of the robotic manipulator 1 determined by the positioning apparatus 3, and the dangerous regions are regions where the surgical instrument 111 is not allowed to perform the surgical operation. In general, during surgery, the surgical instrument 111 can only move within a specified range and angle, which can ensure that the surgical instrument 111 performs the surgical operation only in the specified region, and does not enter the dangerous regions.

At a fourth step, the control apparatus 2 may plan an action that the robotic manipulator 1 needs to perform, and further control the surgical instrument 111 carried by the robotic manipulator 1 to perform the surgical operation. A positioning mark may be arranged on the robotic manipulator 1, and in particular, at least one first positioning mark 13 may be arranged on the surgical instrument 111, so that the positioning apparatus can determine the position of the surgical instrument 111 by acquiring the position of the first positioning mark 13 on the surgical instrument 111, monitoring the behavior and action of the surgical instrument 111 in real time.

In an implementation, the surgical instrument assembly 11 is provided with multiple surgical instruments 111, each of which can be provided with the first positioning mark 13. In practical applications, different surgical instruments 111 may be provided with different first positioning marks 13, so that it is possible for the positioning apparatus to distinguish which surgical instrument 111 each first positioning marker 13 corresponds to when acquiring the positions of multiple first positioning marks 13.

At a fifth step, on the premise that the control apparatus 2 determines the surgical path and can acquire the position of the robotic manipulator 1 collected by the positioning apparatus arranged on the robotic manipulator 1, the planning apparatus built in the control apparatus 2 can plan the action that needs to be performed by the robotic manipulator 1.

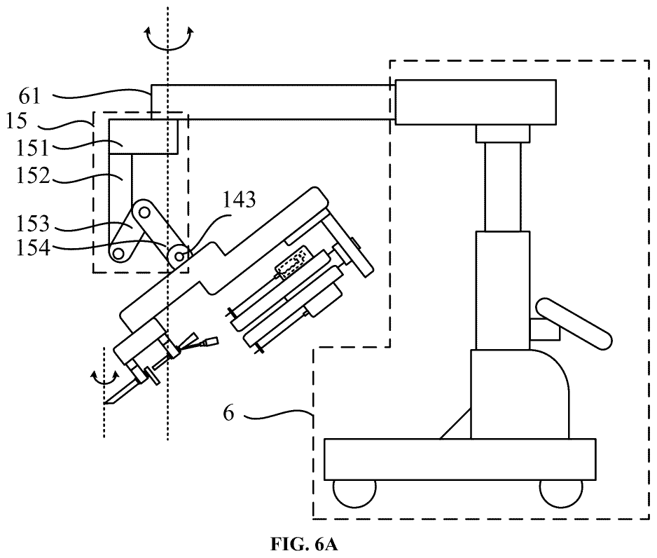

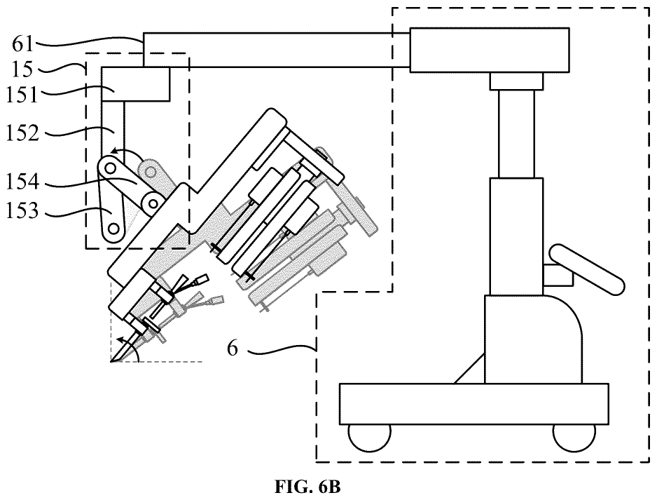

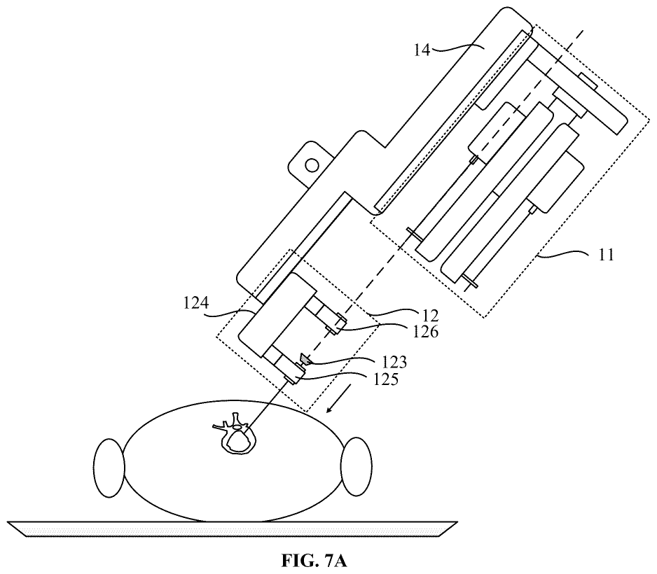

Specifically, in the surgical device provided in the present specification, the robotic manipulator 1 may have multiple degrees of freedom. Therefore, in one or more embodiments of the present specification, the robotic manipulator 1 can change positions and poses of the surgical instrument assembly 11 and the auxiliary instrument assembly 12 mounted on the robotic manipulator 1 by adjusting the position and pose of the robotic manipulator 1 in response to a swing instruction sent by the control apparatus 2. Taking the robotic manipulator 1 being mounted on the base 6 in a suspension manner as an example, as shown in FIG. 6A, the surgical device further includes the base 6 and the suspension platform 61, where the head end of the suspension platform 61 is mounted on the base 6; and the robotic manipulator 1 is hoisted at the tail end of the suspension platform 61.

In an implementation, the robotic manipulator 1 further includes a swing mechanism 15, and the swing mechanism 15 includes at least a first manipulator 151, a second manipulator 152, a third manipulator 153, and a fourth manipulator 154; the robotic manipulator 1 is hoisted at the tail end of the suspension platform 61 through the swing mechanism 15.

A first end of the first manipulator 151 is fixed to the tail end of the suspension platform 61, and a second end of the first manipulator 151 is rotatably connected to a first end of the second manipulator 152; a first end of the third manipulator 153 is connected to a second end of the second manipulator 152, and a second end of the third manipulator 153 is rotatably connected to a first end of the fourth manipulator 154; a second end of the fourth manipulator 154 is rotatably connected to the rotating member 143 arranged on the fixed base 14 of the robotic manipulator 1.

As shown in FIG. 6A, in response to a first swing instruction sent by the control apparatus 2, the robotic manipulator 1 controls the first manipulator 151 to rotate around the tail end of the suspension platform 61, driving the swing mechanism 15 and the robotic manipulator 1 connected to the swing mechanism 15 to rotate, so as to drive the surgical instrument 111 included in the surgical instrument assembly 11 arranged on the fixed base 14 to rotate with the tail end of the surgical instrument 111 as the rotation center, and drive the working cannula 121 included in the auxiliary instrument assembly 12 arranged on the fixed base 14 to rotate with the tail end of the working cannula 121 as the rotation center.

As shown in FIG. 6B, in response to a second swing instruction sent by the control apparatus 2, the robotic manipulator 1 controls the third manipulator 153 to rotate around the second end of the second manipulator 152, driving the fourth manipulator 154 connected to the third manipulator 153 to move, so as to drive the robotic manipulator 1 connected to the fourth manipulator 154 to move, which causes the surgical instrument 111 included in the surgical instrument assembly 11 mounted on the fixed base 14 to swing around the tail end of the surgical instrument, and drives the working cannula 121 included in the auxiliary instrument assembly 12 arranged on the fixed base 14 to swing around the tail end of the working cannula 121.

In the above solutions, the first swing instruction and the second swing instruction may be generated and sent by the control apparatus 2 according to the position of the robotic manipulator 1, the connection relationship between the robotic manipulator 1 and the suspension platform 61, and the mounting position of the surgical instrument assembly 11 on the robotic manipulator 1. Through the above solutions, the robotic manipulator 1 can control itself to move to the spatial position indicated by the first swing instruction and the second swing instruction, so as to make the axis of the surgical instrument 111 that needs to perform the surgical operation in the surgical instrument assembly 11 coincide with the surgical path.

It should be noted that the above swing process of the robotic manipulator 1 is described merely by taking the robotic manipulator 1 being mounted on the base 6 in a suspension manner as an example, which does not represent that the mounting manner of the robotic manipulator 1 is merely the suspension. The robotic manipulator 1 may be mounted on the base 6 in a floor manner. A specific mounting manner may be determined according to a specific application scenario. The mounting manner in which the robotic manipulator 1 is mounted on the base 6 is not limited in this specification.

In an implementation, since the surgical instrument 111 is mounted on the robotic manipulator 1, the purpose of limiting the movement of the surgical instrument 111 only within a specified range and angle can be achieved by limiting the swing of the robotic manipulator 1, so as to ensure that the surgical instrument 111 on the robotic manipulator 1 cannot leave the specified region, and further ensure the safety of surgery.

At a sixth step, the control apparatus 2 may send a control instruction to the robotic manipulator 1 according to the determined surgical path planned for the diseased part, so that the robotic manipulator 1, in response to the control instruction, performs the surgical operation on the specified region. In the embodiments of the present specification, the robotic manipulator 1, in a case of being provided with the surgical instrument assembly 11, is further provided with the auxiliary instrument assembly 12, so that the robotic manipulator 1 can not only perform the surgical operation through the surgical instrument 111 in the surgical instrument assembly 11, but also perform preoperative assistance and intraoperative assistance during the surgical operation through the auxiliary instrument assembly 12. The preoperative assistance may include actions such as puncturing, mounting the working cannula 121, and controlling the working cannula 121 to perform tilting, rotating and the like; the intraoperative assistance may include actions such as controlling the working cannula 121 to perform tilting, rotating and the like, and controlling the surgical instrument 111 to perform advancing, retracting, rotating, tilting, surgical operation and the like.

Specifically, the robotic manipulator 1 may include the fixed base 14, and the auxiliary instrument assembly 12 and the surgical instrument assembly 11 may be arranged on the fixed base 14.

The auxiliary instrument assembly 12 includes a first auxiliary sliding block 124, a first auxiliary clamping apparatus 125, and a second auxiliary clamping apparatus 126; the first auxiliary sliding block 124 slides on the fixed base 14, and the first auxiliary clamping apparatus 125 and the second auxiliary clamping apparatus 126 are respectively slidably connected to the first auxiliary sliding block 124; the first auxiliary sliding block 124 slides on the fixed base 14 to drive the first auxiliary clamping apparatus 125 and the second auxiliary clamping apparatus 126 to slide along an extending direction of the fixed base 14, so that tools clamped by the first auxiliary clamping apparatus 125 and the second auxiliary clamping apparatus 126 slide along the extending direction of the fixed base 14.

The tools clamped by the first auxiliary clamping apparatus 125 and the second auxiliary clamping apparatus 126 may be a puncturing tool 123, a working cannula 121, an endoscope 122, etc. Processes of the first auxiliary clamping apparatus 125 clamping the puncturing tool 123 to perform puncturing and clamping the working cannula 121 to establish a working channel, and the second auxiliary clamping apparatus 126 clamping the endoscope 122 will be described in detail below respectively.

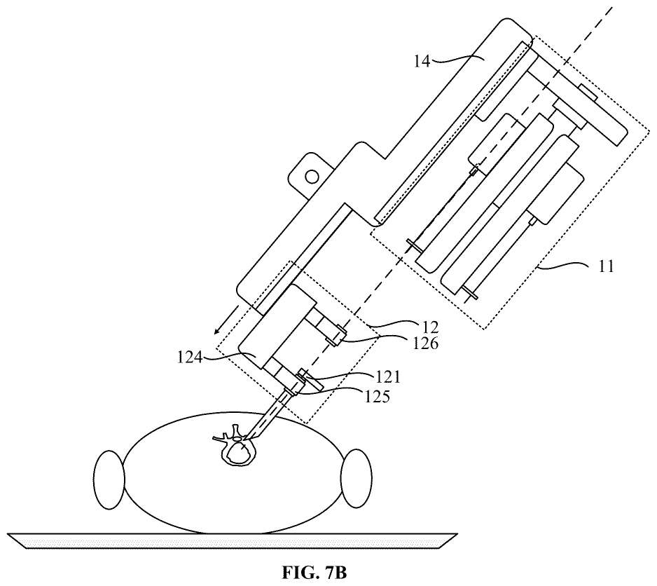

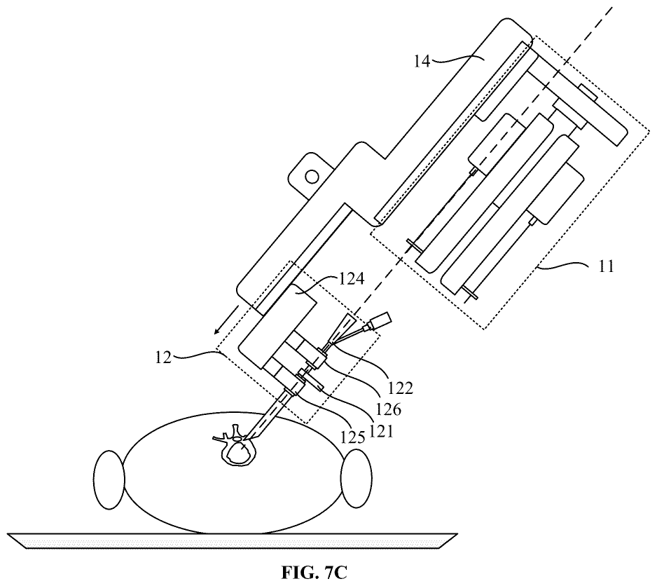

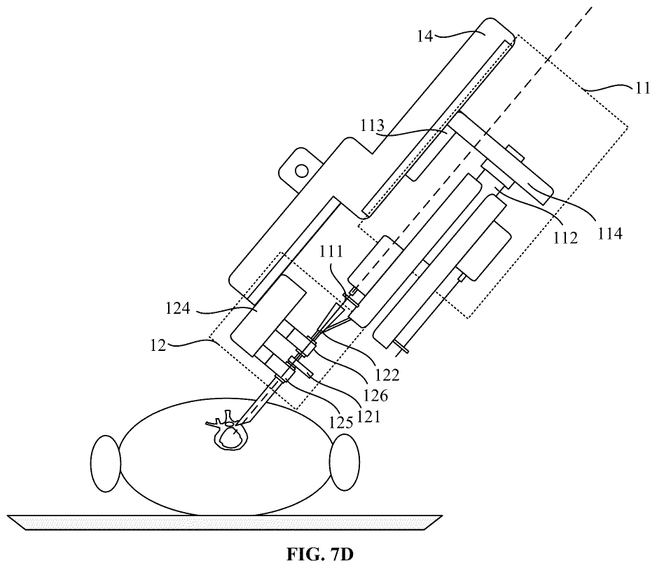

For the process of the first auxiliary clamping apparatus 125 clamping the puncturing tool 123 to puncture the patient, as shown in FIG. 7A, when the patient is located on the surgical table and the robotic manipulator 1 swings to a position suitable for the surgical operation, the first auxiliary clamping apparatus 125 clamps the puncturing tool 123 to perform puncturing from a pre-planned puncturing position along a pre-planned puncturing direction in response to a puncturing instruction sent by the control apparatus 2, so that the head end of the puncturing tool 123 reaches the specified region and a puncturing wound is formed. Since instruments such as the working cannula 121 need to be inserted through the puncturing wound in subsequent surgical operations, the puncturing wound may be obtained by a reaming tool reaming a puncturing channel obtained by the puncturing tool 123 performing the puncturing after the puncturing tool 123 performs the puncturing. Of course, the puncturing tool 123 itself may also have puncturing and reaming functions. The puncturing tool 123 used in the embodiments of the present specification may be any currently existing puncturing tool 123 with puncturing and reaming functions. The specific material and the puncturing and reaming manner of the puncturing tool 123 are not limited in this specification.

Further, for the puncturing wound formed by puncturing and reaming, the operator may replace the puncturing tool 123 with the working cannula 121, or when withdrawing the puncturing tool 123, leave its outermost working cannula 121. As shown in FIG. 7B, the working cannula 121 is inserted into the puncturing wound along the puncturing direction, so that the head end of the working cannula 121 is located in the specified region.

For the process of the second auxiliary clamping apparatus 126 clamping the endoscope 122 and placing the endoscope 122 into the puncturing wound, as shown in FIG. 7C, after the working cannula 121 is placed, the endoscope 122 may be clamped by the second auxiliary clamping apparatus 126, and in response to an endoscope control instruction sent by the control apparatus 2, the first auxiliary sliding block 124 slides on the fixed base 14 to drive the endoscope 122 to be inserted into the working cannula 121. The endoscope 122 may collect a real-time image of the specified region and send the real-time image of the specified region to the control apparatus 2, so that the operator performs the surgical operation on the specified region through the control apparatus 2 according to the real-time image of the specified region.

It can be understood that, for the processes of the first auxiliary clamping apparatus 125 and the second auxiliary clamping apparatus 126 respectively clamping different instruments, each has 3 or more degrees of freedom, that is, the actions of the instruments, such as advancing, retracting, tilting, and rotating can be completed. The actions performed by multiple auxiliary instruments included in the auxiliary instrument assembly 12 may be determined according to an operator and a practical application scenario, which is not limited in this specification.

At a seventh step, after the working channel is established through the working cannula 121, and the endoscope 122 is placed in the working cannula 121, it is possible to control the sliding block 113 to slide on the fixed base 14 and/or the clamping apparatus 114 to slide on the sliding block 113, so that the surgical instrument 111 required for the surgical operation enters the instrument channel in the endoscope 122, as shown in FIG. 7D, to reach the specified region to perform the surgical operation. In this process, it is necessary to determine whether the surgical instrument 111 currently located at the operation position is the specified surgical instrument 111 required for the surgical operation. If the surgical instrument 111 currently located at the operation position is the specified surgical instrument 111 required for the surgical operation, the control apparatus 2 may directly generate an instrument moving instruction, so that the robotic manipulator 1 controls the specified surgical instrument 111 to move to the specified region along the surgical path. If the surgical instrument 111 currently located at the operation position is not the specified surgical instrument 111 required for the surgical operation, it is necessary to determine a to-be-selected position where the specified surgical instrument 111 required for the surgical operation is located, determine the control parameter of the switching apparatus 112 according to the to-be-selected position of the specified surgical instrument 111 and the operation position, and generate the instrument switching instruction according to the control parameter, so as to move the specified surgical instrument 111 to the operation position, where the control parameter includes at least one of a moving direction, a moving distance or a rotation angle.

At an eighth step, during surgery, since different surgical operations can be performed through different surgical instruments 111, after the surgical instrument 111 required for the current surgical operation completes the current surgical operation, the robotic manipulator 1 may control the surgical instrument 111 to return to a specified position where surgical switching can be performed, and after switching to the specified surgical instrument 111 required for the next surgical operation, it may move to the surgical position again to continue with the next surgical operation.

In this process, the control apparatus 2 can first determine whether the surgical instrument 111 completes the surgical operation according to the current action performed by the surgical instrument 111 required for the current surgical operation; if the surgical instrument 111 does not complete the surgical operation, wait for the surgical instrument 111 to complete the surgical operation; if the surgical instrument 111 completes the surgical operation, generate a return instruction to move the surgical instrument 111 to the specified position. The specified surgical instrument 111 required for the next surgical operation is determined, and the instrument switching instruction is generated according to the to-be-selected position where the specified surgical instrument 111 required for the next surgical operation is located and the operation position, so as to move the specified surgical instrument 111 required for the next surgical operation to the operation position. Further, the instrument moving instruction is generated again to control the specified surgical instrument 111 currently located at the operation position to move to the surgical position and perform the surgical operation.

In practical applications, the eighth step may be repeated multiple times to achieve the purpose of using multiple different surgical instruments 111 to complete multiple different operations in this surgical process.

In an embodiment of the present specification, the surgical device provided in the present specification may implement master-slave control through the control instruction generated by the control apparatus 2 in response to operations of the operator.

Specifically, the control apparatus 2 further includes a display 22 and a human-computer interaction unit 21.

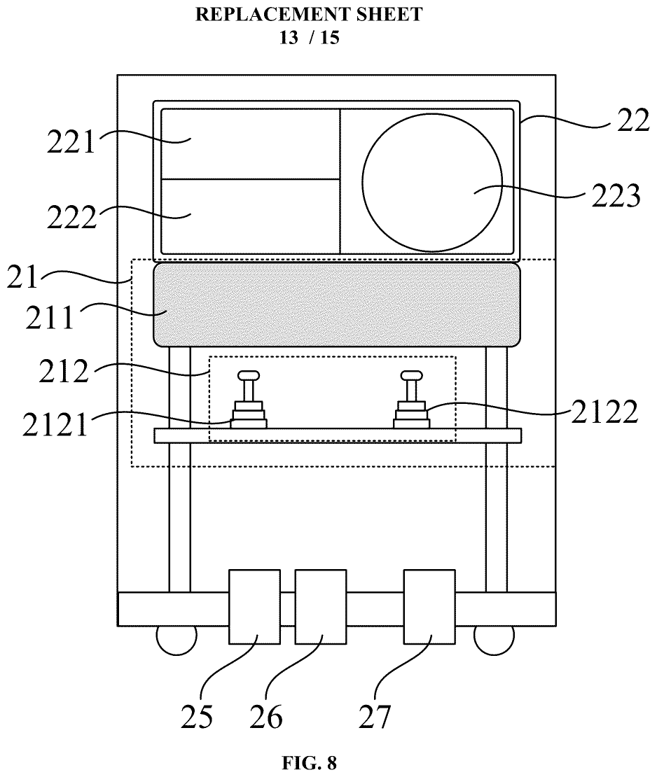

The display 22 can show for assisting the operator in controlling the robotic manipulator 1 and show the image used. By way of human-computer interaction with the human-computer interaction unit 21, the operator can control the robotic manipulator 1 to move, control the auxiliary instrument assembly 12 to implement actions of puncturing, placing and rotating the working cannula 121, and placing the endoscope 122, and control the surgical instrument 111 in the surgical instrument assembly 11 to perform the surgical operation, etc. FIG. 8 is a schematic diagram of a control apparatus provided in the present specification.

Specifically, the display 22 mounted on the control apparatus 2 is configured to display the scanning result (a CT or X-ray image) of the diseased part of the patient, the three-dimensional image generated by the control apparatus 2, and the real-time image of the diseased part collected by the endoscope 122, or may further additionally display more information, such as an Electrocardiogram (ECG) or electrophysiological monitoring information of the patient, so as to provide reference for the operator to perform surgery. As shown in FIG. 8, the scanning result of the diseased part of the patient may be displayed in the display region 221, the three-dimensional image generated by the control apparatus 2 may be displayed in the display region 222, and the real-time image of the diseased part in a wound on a patient body collected by the endoscope 122 may be displayed in the display region 223. The three-dimensional image generated by the control apparatus 2 shown in the display region 222 may be a three-dimensional image of the endoscope 122, the surgical instrument 111 and the diseased part of the patient simulated by the control apparatus 2 through the built-in specified three-dimensional software.

The human-computer interaction unit 21 may include a touch screen 211 and/or a control rod 212, where the control rod 212 may include an auxiliary control rod 2121 and an operation control rod 2122.

The auxiliary control rod 2121 can be controlled to rotate, press, tilt toward gear actions in four different directions of front, back, left and right, etc., so as to generate different control instructions for the endoscope 122 in the auxiliary instrument assembly 12 to control the endoscope 122 to implement actions of moving, rotating or tilting.

The operation control rod 2122 can also be controlled to rotate, press, tilt toward gear actions in four different directions of front, back, left and right, etc., so as to generate different control instructions for the surgical instrument located at the operation position in the surgical instrument assembly 11 to control the surgical instrument 111 to implement actions of moving, rotating, tilting and surgical operations.

Further, the operator can input an operation action through the auxiliary control rod 2121 and/or the operation control rod 2122 by referring to the image on the display 22, and generate different types of control instructions according to different operation actions of different control rods.

In an implementation, the control rod may be divided into several sub-control rods according to different control functions, and the several sub-control rods are respectively arranged in the control apparatus 2, so that operation actions originally input through the single control rod are distributed to different sub-control rods to complete. Due to the increase in the number of the control rods, the complexity of operations is increased, but, since each sub-control rod can correspond to a single instrument action, the difficulty of master-slave control operations is reduced, and the efficiency of surgical operations is still improved to a certain extent.

In addition, it is also possible to provide an auxiliary foot brake 25, an operation foot brake 26 and an emergency stop foot brake 27 to cooperate with the operations of the auxiliary control rod 2121 and the operation control rod 2122.

Specifically, when the operator steps on the auxiliary foot brake 25, the operator can control the auxiliary control rod 2121 to finely adjust the angle and position of the light source and the camera at the tail end of the endoscope 122. If the operator does not step on the auxiliary foot brake 25, even if the operator operates the auxiliary control rod 2121, no operations can be performed to the endoscope 122.

When the operator steps on the operation foot brake 26, the operator can control the operation control rod 2122 to operate the surgical instrument 111, adjust the position of the surgical instrument 111 on the patient body, implement fine adjustments of the angle and position of the surgical instrument 111, and control the surgical instrument 111 to complete corresponding surgical operations. If the operator does not step on the operation foot brake 26, even if the operator operates the operation control rod 2122, no operations can be performed, so as to ensure safety.

In addition, the control apparatus 2 is further provided with the emergency stop foot brake 27. Once the operator finds that an abnormal operation occurs on the robotic manipulator 1 and/or during the execution of a puncturing instruction, a rotating instruction or a surgical operation instruction, and an abnormal operation occurs on the robotic manipulator 1 and/or at the time of the operator performing a manual operation through the control apparatus 2, the operator can step on the emergency stop foot brake 27 to stop the movement of the robotic manipulator 1.

Of course, the operator can also manually make fine adjustments to the angles and positions of the endoscope 122 and the surgical instrument 111 within a wound according to a specific surgical situation, and manually operate the surgical instrument 111 to perform surgical treatment on the diseased part, which is not limited in this specification.

In an embodiment of the present specification, intraoperative CT or C-arm X-ray scanning may be performed on the patient during surgery to obtain an intraoperative X-ray image of the specified region within the diseased part of the patient. Since intraoperative CT or C-arm X-ray scanning causes certain radiation in the surgical environment, in order to protect the physical safety of the operator, a protective apparatus may be placed at the periphery of the control apparatus 2, for example, the protective apparatus may be a ray protective plate or a lead glass apparatus with an equivalent of >=0.5 mmPb, so as to prevent the operator from being exposed to radiation in the surgical environment.

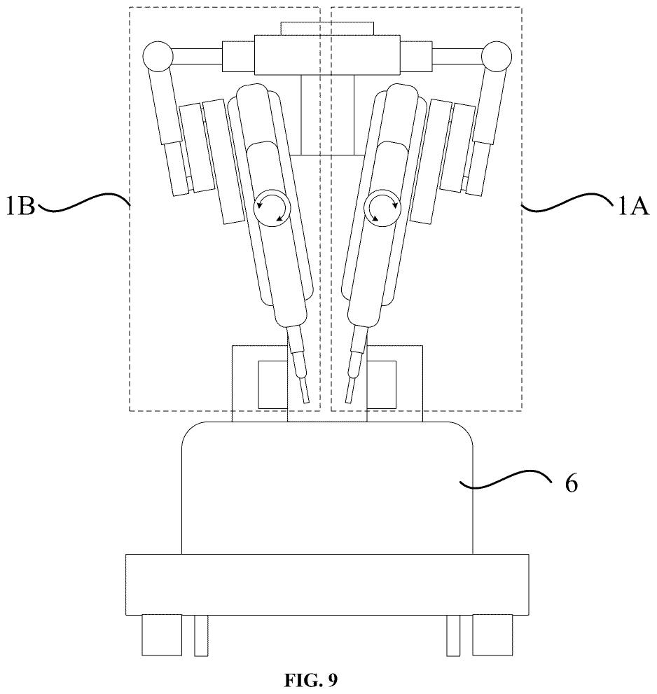

In an embodiment of the present specification, the base 6 may be provided with an operation robotic manipulator 1A and an auxiliary robotic manipulator 1B, as shown in FIG. 9, where the operation robotic manipulator 1A is provided with the surgical instrument assembly 11, and the auxiliary robotic manipulator 1B is provided with the auxiliary instrument assembly 12, in which way, a surgical device that can perform UBE surgery is formed. The auxiliary robotic manipulator 1B is provided with the auxiliary instrument assembly 12, and the working cannula 121 and the endoscope 122 are sequentially placed into the patient by controlling the auxiliary robotic manipulator 1B, so as to collect a real-time image of the specified region of the patient and send the real-time image to the control apparatus 2. A surgical instrument 111 included in the surgical instrument assembly 11 arranged on the operation robotic manipulator 1A enters the patient body from another puncturing wound different from the puncturing wound where the working cannula 121 is located, and reaches the specified region to perform the surgical operation on the specified region in response to a pre-planned surgical action or an operation action performed by an operator through the control apparatus 2.

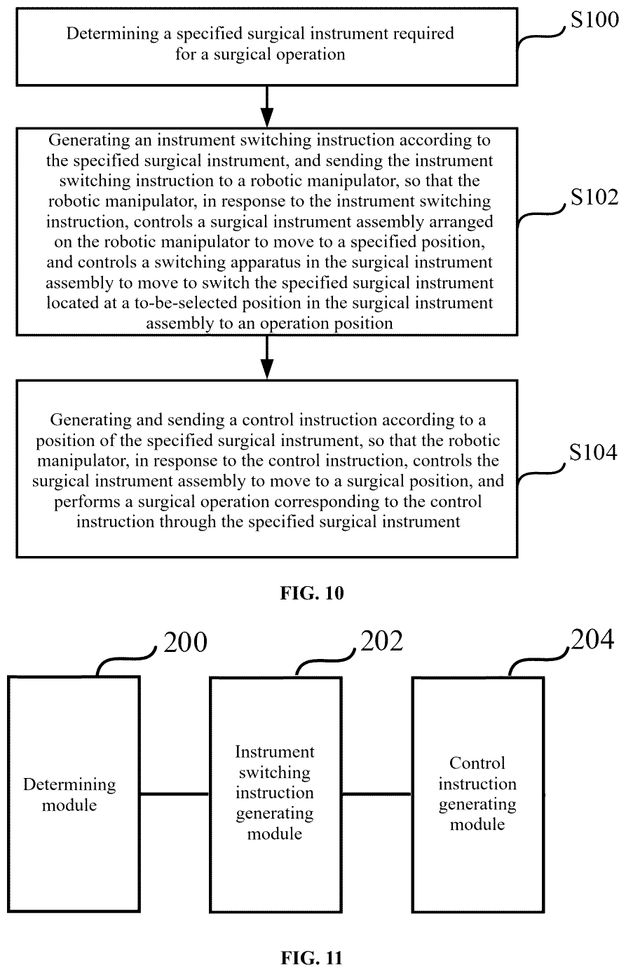

In order to facilitate understanding of the above surgical device, the present specification further provides a schematic flowchart of a surgical method, to illustrate an operating method of the surgical device in an actual surgical process, as shown in FIG. 10.

FIG. 10 is a schematic flowchart of an operating method of a minimally invasive surgical device provided in the present specification, including the following steps.

S100: determining a specified surgical instrument required for a surgical operation.

Generally, a control apparatus may be a computer host equipped with specified software (such as three-dimensional software, and planning software), and is connected to apparatuses deployed in a surgical environment, such as a robotic manipulator, a positioning apparatus or a scanning apparatus 4, in a wired or wireless manner. According to the surgical method provided in the embodiments of the present specification, an execution process of the surgical method may be performed by the control apparatus.

In practical applications, after a patient is in position, the robotic manipulator reaches a predetermined position, a working cannula establishes a working channel, and an endoscope is placed in the working cannula, a surgical instrument is operated to enter a diseased part of the patient to perform a surgical operation. Before the surgical instrument is controlled to enter the diseased part, it is necessary to determine whether the surgical instrument located at the operation position is the surgical instrument required for the current surgical operation. If the surgical instrument located at the operation position is the surgical instrument required for the current surgical operation, the control apparatus may generate an instrument control instruction to complete the surgical operation. If the surgical instrument located at the operation position is not the surgical instrument required for the current surgical operation, the specified surgical instrument required for the surgical operation needs to be determined and switched to the operation position.

In addition, in the embodiments of the present specification, the determining manner of the specified surgical instrument required for the surgical operation may be based on a use sequence of surgical instruments pre-stored in the control apparatus, or it may be determined by an operator through the human-computer interaction unit arranged in the control apparatus, which is not limited in this specification.

S102: generating an instrument switching instruction according to the specified surgical instrument, and sending the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position.

After the specified surgical instrument is determined, the instrument switching instruction may be generated according to the to-be-selected position where the specified surgical instrument is located and the operation position, and the instrument switching instruction may include a control parameter of the switching apparatus.

S104: generating and sending a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

After it is determined that the specified surgical instrument is located at the operation position, the control apparatus sends the control instruction to the robotic manipulator, so that the robotic manipulator, in response to the control instruction sent by the control apparatus, controls the surgical instrument included in the surgical instrument assembly arranged on the robotic manipulator to perform the surgical operation on the specified region.

Based on the surgical method shown in FIG. 10, the control apparatus sends the control instruction to the robotic manipulator according to the positions of the robotic manipulator and the specified region and a first operation action of the operator,, so that the robotic manipulator, in response to the control instruction, controls the surgical instrument included in the surgical instrument assembly to perform the surgical operation on the specified region, and the control apparatus sends the instrument switching instruction to the robotic manipulator in response to a second operation action of the operator, so that the robotic manipulator, according to the instrument switching instruction, controls switching between surgical instruments arranged on the switching apparatus. It can be seen that rapid switching between multiple surgical instruments is realized by the surgical device, which can eliminate the operations for operators to manually replace the surgical instruments during surgery in surgical scenarios requiring a large number of surgical instruments, further improving the precision of surgical operations and relieving the operation burden on operators.

The above is the surgical method provided in one or more embodiments of the present specification. Based on the same idea, the present specification further provides a corresponding surgical apparatus, as shown in FIG. 11.

FIG. 11 is a schematic diagram of a surgical apparatus provided in the present specification, specifically including:
a determining module 200, configured to determine a specified surgical instrument required for a surgical operation;
an instrument switching instruction generating module 202, configured to generate an instrument switching instruction according to the specified surgical instrument, and send the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position; and
a control instruction generating module 204, configured to generate and send a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

The present specification further provides a computer-readable storage medium, where the storage medium stores a computer program, and the computer program can be used to perform the surgical method provided in FIG. 10.

Of course, in addition to the software implementation manner, the present specification does not exclude other implementation manners, such as manners of logic devices or combination of software and hardware, that is, the executing subject of the following processing flow is not limited to logic units, and may also be the hardware or the logic devices.

In the 1990s, improvements to a technology may be clearly distinguished as either improvements in hardware (for example, improvements to circuit structures such as diodes, transistors, and switches) or improvements in software (improvements to method flows). However, with the development of technologies, improvements to many method flows today can be already deemed as direct improvements to hardware circuit structures. Designers almost always obtain corresponding hardware circuit structures by programming improved method flows into hardware circuits. Thus, it cannot be said that improvements to a method flow cannot be implemented with hardware entity modules. For example, a Programmable Logic Device (PLD) (for example, a Field Programmable Gate Array (FPGA)) is such an integrated circuit, whose logic functions are determined by a user programming the device. A designer programs by himself/herself to “integrate” a digital system on a piece of PLD, without needing for a chip manufacturer to design and manufacture a dedicated integrated circuit chip. Moreover, nowadays, instead of manually manufacturing an integrated circuit chip, such programming is also mostly implemented with “logic compiler” software, which is similar to a software compiler used in program development and writing, and original codes before compilation also need to be written in a specific programming language, which is referred to as Hardware Description Language (HDL). There is not only one type of HDL, but there are many types of HDL, such as ABEL (Advanced Boolean Expression Language), AHDL (Altera Hardware Description Language), Confluence, CUPL (Cornell University Programming Language), HDCal, JHDL (Java Hardware Description Language), Lava, Lola, MyHDL, PALASM, and RHDL (Ruby Hardware Description Language). Currently most commonly used are VHDL (Very-High-Speed Integrated Circuit Hardware Description Language) and Verilog. It should also be clear to those skilled in the art that, only by logically programming a method flow with the above several hardware description languages simply and programming it into an integrated circuit, a hardware circuit that implements the logic method flow can be easily obtained.

A controller can be implemented in any suitable manner, for example, the controller may take the form of a microprocessor or processor and a computer-readable medium that stores computer readable program codes (for example, software or firmware) executable by the (micro) processor, a logic gate, a switch, an Application Specific Integrated Circuit (ASIC), a programmable logic controller, and an embedded microcontroller. Examples of the controller include, but are not limited to, the following microcontrollers: ARC 625D, Atmel AT91SAM, Microchip PIC18F26K20, and Silicone Labs C8051F320. A memory controller may also be implemented as a part of control logic of a memory. It will also be appreciated by those skilled in the art that, in addition to implementing the controller in a manner of pure computer-readable program codes, it is entirely possible to logically program the method steps to enable the controller to implement the same functions in the form of a logic gate, a switch, an application specific integrated circuit, a programmable logic controller, and an embedded microcontroller, etc. Therefore, such a controller can be considered as a hardware component, and apparatuses included therein for implementing various functions can also be considered as structures in the hardware component. Alternatively, even the apparatuses for implementing various functions can be considered as both software modules that implement the method and structures in the hardware component.

The system, apparatus, module or unit set forth in the above examples may be specifically implemented by a computer chip or an entity, or implemented by a product having a certain function. A typical implementation device is a computer. Specifically, the computer may be, for example, a personal computer, a laptop computer, a cellular phone, a camera phone, a smart phone, a personal digital assistant, a media player, a navigation device, an e-mail device, a game control apparatus, a tablet computer, a wearable device, or a combination of any of these devices.

For the convenience of description, during the description of the apparatus, it is divided into various units according to their functions, which are described respectively. Of course, when the present specification is implemented, functions of the units may be realized in one or more software and/or hardware.

Those skilled in the art should understand that the embodiments of the present disclosure may be provided as methods, systems, or computer program products. Accordingly, the present disclosure may take the form of a pure hardware embodiment, a pure software embodiment, or an embodiment combining software and hardware aspects. Furthermore, the present disclosure may take the form of a computer program product implemented on one or more computer usable storage media (including, but not limited to, disk storage, Compact Disc Read-Only Memory (CD-ROM for short), optical storage, etc.) having computer usable program codes therein.

The present disclosure is described with reference to the flowcharts and/or the block diagrams of the methods, the apparatuses (or systems), and the computer program products according to the embodiments of the present disclosure. It should be understood that each process and/or block, and combinations of processes and/or blocks in the flowcharts and/or block diagrams can be implemented by computer program instructions. These computer program instructions may be provided to a processor of a general-purpose computer, a special-purpose computer, an embedded processing machine or other programmable data processing device to produce a machine, so that the instructions executed by the processor of the computer or other programmable data processing device produce an apparatus for realizing functions specified in one or more processes in a flowchart and/or one or more blocks in a block diagram.

These computer program instructions can also be stored in a computer-readable memory capable of directing a computer or other programmable data processing device to operate in a particular manner, so that the instructions stored in the computer-readable memory produce an article of manufacture including an instruction apparatus. The instruction apparatus can realize functions specified in one or more processes in a flowchart and/or one or more blocks in a block diagram.

These computer program instructions can also be loaded on a computer or other programmable data processing device, so that a series of operation steps are performed on the computer or other programmable device to produce processes implemented by a computer, and the instructions executed on the computer or other programmable device provide steps for realizing functions specified in one or more processes in a flowchart and/or one or more blocks in a block diagram.

In a typical configuration, a computing device includes one or more processors (CPUs), an input/output interface, a network interface and a memory.

The memory may include a non-permanent memory in a computer-readable medium such as a random access memory (RAM) and/or a non-volatile memory such as a read-only memory (ROM) or a flash RAM. The memory is an example of the computer-readable medium.

Computer-readable media include permanent and non-permanent, removable and non-removable media. Any method or technology can be used to implement information storage. The information may be computer readable instructions, data structures, modules of programs, or other data. Examples of storage media of a computer include, but are not limited to, phase-change memory (PRAM), static random access memory (SRAM), dynamic random access memory (DRAM), other types of random access memory (RAM), read-only memory (ROM), electrically erasable programmable read-only memory (EEPROM), flash memory or other memory technology, Compact Disc Read-Only Memory (CD-ROM), digital versatile disc (DVD) or other optical storage, magnetic cassette, a magnetic tape disk storage or other magnetic storage device or any other non-transmission medium which can be used to store information that can be accessed by the computing device. According to the definitions herein, the computer-readable medium does not include transitory computer-readable media (transitory media) such as a modulated data signal and carrier wave.

It shall be further noted that the term “including”, “containing” or any variation thereof is intended to encompass non-exclusive inclusion, so that a process, method, article or device including a series of elements includes not only those elements but also other elements not listed explicitly or those elements inherent to such process, method, article or device. Without more limitations, an element defined by the statement “including a . . . ” does not exclude the existence of other identical elements in the process, method, commodity or device that includes the element.

Those skilled in the art should understand that the embodiments of the present specification may be provided as methods, systems or computer program products. Accordingly, the present specification may take the form of a pure hardware embodiment, a pure software embodiment, or an embodiment combining software and hardware aspects. Furthermore, the present specification may take the form of a computer program product implemented on one or more computer usable storage media (including, but not limited to, disk storage, CD-ROM, optical storage, etc.) having computer usable program codes therein.

The present specification may be described in the general context of computer-executable instructions executed by a computer, such as program modules. Generally, the program modules include routines, programs, objects, components, data structures, and so forth that perform particular tasks or implement particular abstract data types. The present specification may also be practiced in distributed computing environments. In these distributed computing environments, tasks are performed by remote processing devices connected through a communication network. In a distributed computing environment, the program modules may be located in both local and remote computer storage media including storage devices.

Various embodiments in the present specification are described in a progressive manner. Same or similar parts in the various embodiments may be referred to for each other, and each embodiment focuses on the differences from other embodiments. Especially, for system embodiments, since they are basically similar to the method embodiments, the description is simplified, and reference may be made to some of the description of the method embodiments.

The above are only embodiments of the present specification, which are not intended to limit the present specification. For those skilled in the art, the present specification can have various modifications and variations. Any modification, equivalent replacement, improvement, etc. made within the spirit and principle of the present specification should be included within the scope of the claims of this specification.

1. A surgical device, comprising a robotic manipulator (1) and a control apparatus (2), wherein the robotic manipulator (1) is provided with a surgical instrument assembly (11) comprising multiple surgical instruments (111) arranged on a switching apparatus (112);
the control apparatus (2) is configured to:
determine a specified surgical instrument (111) required for a surgical operation, generate an instrument switching instruction according to the specified surgical instrument (111), and send the instrument switching instruction to the robotic manipulator (1); and
generate and send a control instruction according to a position of the specified surgical instrument (111);
the robotic manipulator (1) is configured to:
in response to the instrument switching instruction sent by the control apparatus (2), control the surgical instrument assembly (11) to move to a specified position, and control the switching apparatus (112) to move to switch the specified surgical instrument (111) located at a to-be-selected position in the surgical instrument assembly (11) to an operation position; and
in response to the control instruction sent by the control apparatus (2), control the surgical instrument assembly (11) to move to a surgical position, and perform a surgical operation corresponding to the control instruction through the specified surgical instrument (111).

2. The surgical device according to claim 1, wherein a switching manner in which the switching apparatus (112) switches the specified surgical instrument (111) from the to-be-selected position to the operation position comprises at least one of rotation or translation.

3. The surgical device according to claim 1, wherein the switching apparatus (112) further comprises an operation component (1121);
in response to an operation action input by an operator through the operation component (1121), the switching apparatus (112) is controlled to move.

4. The surgical device according to claim 1, wherein the robotic manipulator (1) is further provided with an auxiliary instrument assembly (12) comprising at least a working cannula (121) and an endoscope (122);
a mounting position of the working cannula (121) on the robotic manipulator (1) is located on a first axis, a mounting position of the endoscope (122) on the robotic manipulator (1) is located on a second axis, and a mounting position of the surgical instrument (111) on the robotic manipulator (1) is located on a third axis;
the first axis, the second axis, and the third axis are coincident or parallel to each other.

5. The surgical device according to claim 4, wherein when the first axis, the second axis, and the third axis are parallel to each other, a distance between any two axes of the first axis, the second axis, and the third axis is not greater than a preset threshold.

6. The surgical device according to claim 1, wherein the switching apparatus (112) is uniformly provided with multiple instrument mounting grooves, each of which is mounted with at least one surgical instrument (111); one of the instrument mounting grooves arranged on the switching apparatus (112) is located at the operation position, and the rest of the instrument mounting grooves are located at to-be-selected positions.

7. The surgical device according to claim 6, wherein the control apparatus (2) is configured to, when the specified surgical instrument (111) and a current surgical instrument (111) are mounted on a same instrument mounting groove, generate the instrument switching instruction according to mounting positions of the specified surgical instrument (111) and the current surgical instrument (111) respectively on the instrument mounting groove, and send the instrument switching instruction to the robotic manipulator (1).

8. The surgical device according to claim 1, wherein the control apparatus (2) is configured to determine a control parameter of the switching apparatus (112) according to the to-be-selected position of the specified surgical instrument (111) and the operation position, and generate the instrument switching instruction according to the control parameter, wherein the control parameter comprises at least one of a moving direction, a moving distance, or a rotation angle.

9. The surgical device according to claim 1, further comprising a positioning apparatus (3), wherein a first positioning mark (13) is arranged on the surgical instrument (111), and a second positioning mark (5) is arranged within a preset range of a diseased part of a patient;
the positioning apparatus (3) is configured to collect positions of the first positioning mark (13) and the second positioning mark (5) and send the positions to the control apparatus;
the control apparatus (2) is configured to determine, according to the position of the second positioning mark (5), the specified position that allows each surgical instrument (111) in the surgical instrument assembly (11) to be switched, and the surgical position that allows each surgical instrument (111) to perform the surgical operation.

10. The surgical device according to claim 9, further comprising a scanning apparatus (4), wherein a third positioning mark (41) is arranged on the scanning apparatus (4);
the scanning apparatus (4) is configured to scan the patient and obtain a scanning result of the diseased part of the patient, and send the scanning result to the control apparatus (2);
the positioning apparatus (3) is configured to collect and send a position of the third positioning mark (41);
the control apparatus (2) is configured to determine a relative position between the scanning apparatus (4) and the patient according to the position of the second positioning mark (5) and the position of the third positioning mark (41); and register the scanning result according to the relative position between the scanning apparatus (4) and the patient.

11. The surgical device according to claim 2, wherein the robotic manipulator (1) further comprises a fixed base (14); the surgical instrument assembly (11) further comprises a sliding block (113) and a clamping apparatus (114); wherein the clamping apparatus (114) clamps the switching apparatus (112), the clamping apparatus (114) is slidably connected to the sliding block (113), and the sliding block (113) is slidably connected to the fixed base (14);
the sliding block (113) is configured to, when sliding on the fixed base (14), drive the clamping apparatus (114) to slide along an extending direction of the fixed base (14) so as to drive the surgical instrument (111) to slide along the extending direction of the fixed base (14);
the clamping apparatus (114) is configured to, when sliding on the sliding block (113), drive the surgical instrument (111) to slide along the extending direction of the fixed base (14).

12. The surgical device according to claim 11, wherein the control apparatus (2) is configured to determine, according to positions of the surgical instrument assembly (11) and a diseased part of a patient, a surgical path for the surgical instrument (111) to perform the surgical operation on the diseased part; generate a moving instruction according to the surgical path, and send the moving instruction to the robotic manipulator (1);
the robotic manipulator (1) is configured to move according to a received moving instruction to enable the extending direction of the fixed base (14) to be parallel to the surgical path, so that the surgical instrument (111) reaches the diseased part along the surgical path.

13. The surgical device according to claim 1, wherein the control apparatus (2) further comprises a human-computer interaction unit (21);
the human-computer interaction unit (21) is configured to, in response to an operation of an operator, determine the specified surgical instrument (111).

14. The surgical device according to claim 13, wherein the human-computer interaction unit (21) comprises at least one of a touch screen (211) or a control rod (212).

15. The surgical device according to claim 1, further comprising a base (6) and a suspension platform (61), wherein a head end of the suspension platform (61) is mounted on the base (6); the robotic manipulator (1) is hoisted at a tail end of the suspension platform (61).

16. The surgical device according to claim 1, further comprising a base (6), wherein the robotic manipulator (1) is mounted on the base (6).

17. A surgical method, wherein the method is applied to a control apparatus and comprises:
determining a specified surgical instrument required for a surgical operation;
generating an instrument switching instruction according to the specified surgical instrument, and sending the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position; and
generating and sending a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

18. A surgical apparatus, comprising:
a processor; and
a memory for storing instructions executable by the processor,
wherein the processor is configured to:
determine a specified surgical instrument required for a surgical operation;
generate an instrument switching instruction according to the specified surgical instrument, and send the instrument switching instruction to a robotic manipulator, so that the robotic manipulator, in response to the instrument switching instruction, controls a surgical instrument assembly arranged on the robotic manipulator to move to a specified position, and controls a switching apparatus in the surgical instrument assembly to move to switch the specified surgical instrument located at a to-be-selected position in the surgical instrument assembly to an operation position; and
generate and send a control instruction according to a position of the specified surgical instrument, so that the robotic manipulator, in response to the control instruction, controls the surgical instrument assembly to move to a surgical position, and performs a surgical operation corresponding to the control instruction through the specified surgical instrument.

19. A non-transitory computer-readable storage medium, wherein the storage medium stores a computer program, and when the computer program is executed by a processor, the method according to claim 17 is implemented.
